(19) United States
(12) Patent Application Publication
(10) Pub. No.: US 2015/0023468 A1
(43) Pub. Date: Jan. 22, 2015

(54) SYSTEM AND METHOD FOR REDUCING A WEIGHT OF AN X-RAY SOURCE

(71) Applicant: General Electric Company, Schenectady, NY (US)

(72) Inventors: Yun Zou, Clifton Park, NY (US); Scott Stephen Zelakiewicz, Niskayuna, NY (US); Fengfeng Tao, Clifton Park, NY (US); Xi Zhang, Ballston Lake, NY (US); Timothy Powers, Harvard, IL (US); Thomas Edmund Sjoberg, Waukesha, WI (US); Marc Weasler, Waukesha, WI (US); Denis Perrillat-Amede, Paris (FR)

(21) Appl. No.: 13/943,857

(22) Filed: Jul. 17, 2013

(51) Int. Cl. A61B 6/00; H05G 1/06

(52) U.S. Cl. CPC A61B 6/4405; H05G 1/06
USPC 378/62; 378/101; 378/102; 378/98

(57) ABSTRACT

A portable x-ray system includes a light weight x-ray head including an x-ray tube and a high voltage (HV) tank, wherein the HV tank comprises a compact voltage multiplier configured to receive a low voltage signal and generate a high voltage signal based on the received low voltage signal. Also, the portable x-ray system includes a carrying case comprising low voltage power electronics coupled to the light weight x-ray head through a low voltage cable, and configured to send the low voltage signal to the light weight x-ray head. In addition, the low voltage power electronics is distributed in a predefined space in the carrying case in such a way that a weight of the light weight x-ray head is counter weighed by a weight of the low voltage power electronics to stabilize the portable x-ray system when the light weight x-ray head is rotated in one or more directions.

### BACKGROUND

Embodiments of the present disclosure relate generally to an x-ray system, and more particularly to a system and a method for reducing a weight of an x-ray source in the x-ray system.

Traditional x-ray imaging systems include an x-ray source and a detector array. The x-ray source generates x-rays that pass through an object under scan. These x-rays are attenuated while passing through the object and are received by the detector array. The detector array includes detector elements that produce electrical signals indicative of the attenuated x-rays received by each detector element. Further, the produced electrical signals are transmitted to a data processing system for analysis, which ultimately produces an image.

Typically, a high voltage is required for an x-ray tube in the x-ray source to emit x-rays. Due to this requirement of high voltage and high power operation, the x-ray source includes heavy components, such as generators, which increase the weight of the x-ray source. In one example, for a 4 kW, 120 kV x-ray system, the weight of the x-ray source will be over 30 lb. Further, since the x-ray tube is mounted on a positioner, a cantilever design is required to support and position the x-ray tube at a required location for medical imaging. Also, the positioner is required to be heavy and stable enough to support the x-ray tube.

In addition, the x-ray source includes a high voltage (HV) generator that is disposed in a generator unit and coupled to the tube in the x-ray head via a HV cable and a HV connector. Since the HV cable is used for providing high voltage potential to the tube, the HV cable is required to be large in diameter and heavy in weight. Also, this HV cable is rigidly coupled between the HV generator and the x-ray head, which in-turn makes it difficult for an operator to move the x-ray head to a desired location/position.

Further, in one of the existing x-ray systems, the power electronics, the x-ray tube, and a collimator are disposed into a single unit to reduce the size of the system. However, the weight of this system will still be very high. For example, for a 4 kW, 120 kV monoblock, the x-ray source weight will be over 30 lb. Also, this x-ray source is difficult to move for scanning as the components are disposed in a single unit.

Thus, there is a need for an improved method and structure for reducing the overall weight and size of the x-ray source. Also, there is a need to improve the weight distribution in the x-ray source to have a flexible positioning of the x-ray source.

### BRIEF DESCRIPTION

In accordance with one embodiment described herein, a portable x-ray system is presented. The portable x-ray system includes a light weight x-ray head comprising an x-ray tube and a high voltage (HV) tank, wherein the HV tank comprises a compact voltage multiplier configured to receive a low voltage signal and generate a high voltage signal based on the received low voltage signal. Also, the portable x-ray system includes a carrying case comprising low voltage power electronics coupled to the light weight x-ray head through a low voltage cable, and configured to send the low voltage signal to the light weight x-ray head. In addition, the low voltage power electronics is distributed in a predefined space in the carrying case in such a way that a weight of the light weight x-ray head is counter weighed by a weight of the low voltage power electronics to stabilize the portable x-ray system when the light weight x-ray head is rotated in one or more directions. Further, the carrying case is configured to support a position of the light weight x-ray head when the light weight x-ray head is rotated in the one or more directions, and store the light weight x-ray head during transportation of the portable x-ray system.

In accordance with a further aspect of the present disclosure, a method is presented. The method includes disposing a high voltage (HV) tank within a light weight x-ray head of a portable x-ray system, wherein the HV tank comprises a compact voltage multiplier configured to receive a low voltage signal and generate a high voltage signal based on the received low voltage signal. Further, the method includes distributing low voltage power electronics in a carrying case in such a way that a weight of the light weight x-ray head is counter weighed by a weight of the low voltage power electronics to stabilize the portable x-ray system when the light weight x-ray head is rotated in one or more directions. Also, the method includes supporting, by a carrying case, a position of the light weight x-ray head when the light weight x-ray head is rotated in the one or more directions. Further, the method includes storing the light weight x-ray head within the carrying case during transportation of the portable x-ray system.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical representation of a x-ray source system, in accordance with aspects of the present disclosure;

FIG. 2 is a block diagram of the x-ray source system, in accordance with aspects of the present disclosure;

FIG. 3 is a diagrammatical representation of the x-ray source system, in accordance with one embodiment of the present disclosure;

FIG. 4 is a diagrammatical representation of the x-ray source system, in accordance with another embodiment of the present disclosure;

FIG. 5 is a diagrammatical representation of the x-ray source system, in accordance with yet another embodiment of the present disclosure;

FIG. 6 is a diagrammatical representation of the x-ray source system coupled to a portable unit, in accordance with aspects of the present disclosure; and

FIG. 7 is a flow chart illustrating a method for balancing a weight of the x-ray source system, in accordance with aspects of the present disclosure.

### DETAILED DESCRIPTION

As will be described in detail hereinafter, various embodiments of exemplary structures and methods for balancing a weight of an x-ray source system are presented. By employing the methods and the various embodiments of the system described hereinafter, the overall weight of the x-ray source system may be substantially reduced. Also, the x-ray source system is flexible to move the x-ray tube to a desired location and/or position for scanning an object.

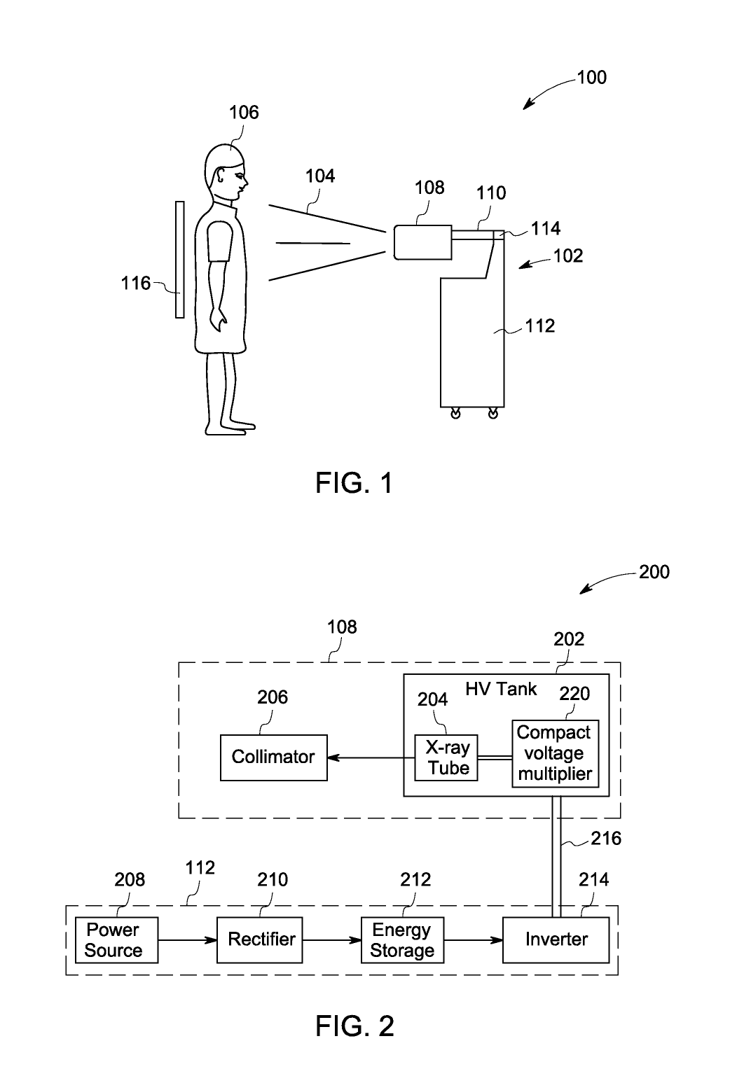

Turning now to the drawings, and referring to FIG. 1, a diagrammatical representation 100 of a portable x-ray source system 102, in accordance with aspects of the present disclosure, is depicted. The portable x-ray source system 102 is configured for emitting x-rays 104 towards a material sample, a patient, or an object 106 that is under scan. The portable x-ray source system 102 includes a light weight x-ray head 108 and a carrying case or base unit 112. In one embodiment, the light weight x-ray head 108 may be rotatable, movable, and/or positional in one or more directions. Further, the carrying case 112 may include an x-ray source support frame 110 that is extended from the carrying case 112 to support the light weight x-ray head 108. Particularly, the x-ray source support frame 110 is used to support a position of the light weight x-ray head 108 when the light weight x-ray head 108 is rotated in one or more directions. It may be noted that the terms “light weight x-ray head” and “head unit” may be used interchangeably in the below description. Also, the terms “carrying case” and “base unit” may be used interchangeably in the below description.

Further, in one embodiment, the x-ray source support frame 110 is rotatably coupled to the base unit 112 so that the head unit 108 may be moved to any desired position for scanning. It may be noted that the terms “x-ray source support frame” and “positioner” may be used interchangeably in the below description. In one example, the positioner 110 may be coupled to the base unit 112 by at least one pivot 114 which aids in rotating or moving the head unit 108 in a desired direction. In one embodiment, the positioner 110 may be included in the base unit 112. More specifically, during transportation of the x-ray source system 102, the base unit 112 may be used as a carrying case that includes the positioner 110 and the head unit 108. Further, when the x-ray source system 102 is used for imaging, the head unit 108 and the positioner 110 may be extended from the carrying case 112, as depicted in FIG. 1.

As will be appreciated, the head unit 108 includes an x-ray tube (not shown) for emitting x-rays towards the patient, or the object 106. The x-ray tube may include a cathode unit and an anode unit that are disposed within an evacuated enclosure. The cathode unit generates an electron beam that is accelerated towards a target surface of the anode unit by applying a high voltage potential between the cathode unit and the anode unit. In one embodiment, an electric current is applied to the electron source, such as a filament, which causes the electron beam to be produced by thermionic emission. The electric current is provided from a high voltage (HV) generator/tank (not shown) that is coupled between the cathode unit and the anode unit.

Further, the electron beam impinges upon the target surface at a focal spot and releases kinetic energy as electromagnetic radiation of very high frequency, i.e., x-rays 104. These x-rays 104 emanate in all directions from the target surface. A portion of these x-rays 104 passes through an outlet of the evacuated enclosure to exit the x-ray tube and be utilized to interact with the object 106. Also, these x-rays 104 are attenuated while passing through the object 106 and are received by a detector 116 causing electrical signals indicative of the attenuated x-rays to be produced. Further, the produced electrical signals are transmitted to a data processing system (not shown) for analysis, which ultimately produces an image.

In accordance with aspects of the present disclosure, the carrying case or base unit 112 includes low voltage power electronics coupled to the head unit 108 through a low voltage cable or a light weight cable. The low voltage power electronics is configured to send a low voltage signal to the head unit 108. In one embodiment, the low voltage signal may include one of a DC voltage and an AC voltage, which is in a range from about 50 V to about 4 kV. The low voltage power electronics may include at least a rectifier (not shown) and an inverter (not shown). The rectifier is used for rectifying a supply voltage received from a power source into a direct current (DC) voltage. Similarly, the inverter is used for converting the DC voltage received from the rectifier into an alternating current (AC) voltage. In one embodiment, the inverter may be disposed within the head unit 108 to balance the weight of the x-ray source system 102. Also, the carrying case or the base unit 112 may include at least one of a battery, a wall plug power regulator, an inverter, and a power source to provide power to the low voltage power electronics. It is to be noted that the working aspects of these components will be explained in greater detail with reference to FIGS. 2-4.

In an exemplary embodiment, the low voltage power electronics may be smartly distributed within a predefined space in the base unit 112. In one embodiment, the low voltage power electronics may be smartly distributed within an available space in the base unit 112 to reduce the size of the base unit 112. In one example, the available space may be a space in the x-ray source support frame 110. Also, the low voltage power electronics may be smartly distributed to balance a weight of the portable x-ray system 102. Particularly, the low voltage power electronics is distributed in the base unit 112 in such a way that a weight of the head unit 108 is counter weighed by a weight of the low voltage power electronics to stabilize the portable x-ray system 102, specifically when the head unit 108 is rotated in one or more directions.

In a conventional x-ray source system, the base unit includes a HV tank or generator in addition to the rectifier and the inverter. Also, the HV tank is coupled to the head unit via a HV cable and a HV connector. Particularly, the HV cable is used for providing a high voltage signal from the base unit to the x-ray tube. To provide such as a high voltage signal, the HV cable should have a diameter or thickness of more than 1 inch and a weight of about 4 lb, which ultimately increases the weight of the X-ray source system. In one example, the weight of the x-ray source system is increased to 100 lb. Also, this HV cable is rigidly coupled between the base unit and head unit, which makes it difficult for an operator to move the head unit to a desired position/location.

To address these problems/shortcomings, in the exemplary system, the HV tank (not shown) is disposed within the head unit 108. Since the HV tank is disposed within the head unit 108, the HV cable and the HV connector is eliminated from the exemplary x-ray source system 102. Also, by disposing the HV tank in the head unit 108, the weight of the x-ray source system 102 may be substantially reduced. This in turn helps the operator to easily move the x-rays source system 102 to a desired location/position. Also, by splitting or distributing the weight, as depicted in FIG. 1, the overall weight of the x-ray source system 102 may be reduced to about 10 lb. In addition, the power electronics, such as the rectifier, the inverter, and other components remaining in the base unit 112 may be equally distributed across the base unit 112 to stabilize and balance the weight of the head unit 108. Moreover, the HV tank includes a compact voltage multiplier that is configured to receive the low voltage signal from the low voltage power electronics and generate a high voltage signal based on the received low voltage signal. In one embodiment, the high voltage signal is in a range from about 10 kV to about 200 kV.

In the presently contemplated configuration, a light weight cable is coupled between the head unit 108 and the base unit 112. Particularly, the light weight cable is used to couple the inverter unit in the base unit 112 to the HV tank in the head unit 108. In one example, the light weight cable may have a diameter in a range from about 0.125 inches to about 0.25 inches and a weight in a range from about 0.1 lb to about 0.5 lb. The light weight cable may be used to carry a high frequency and low voltage signal when compared to the conventional HV cable. The low voltage signal may be in a range from about 50 V to about 4 kV. In one example, the light weight cable may be used to carry 400 V and 110 kHz voltage signal. In one embodiment, the light weight cable may also include one or more wires that are used for carrying tube filament heating current. In one example, the one or more wires may carry the tube filament heating current that is in a range from about 0.5 A to about 5A and voltage that is in a range from about 5 V to about 20 V. It may be noted that the terms “light weight cable” and “low voltage cable” may be used interchangeably in the below description.

Also, the light weight cable is flexible and easily movable, which helps the operator to move the head unit 108 to a desired location/position. In one example, the light weight cable may be disposed within the positioner 110 that is coupled between the head unit 108 and the base unit 112. In addition, the thickness of the light weight cable may be optimized depending upon the frequency and the voltage supplied to the HV tank. The working of the HV tank will be explained in greater detail with reference to FIGS. 2-6. Further, the HV tank may be coupled to the x-ray tube. In one embodiment, the HV tank may be directly coupled to the x-ray tube without any cable and/or additional insulator. For example, the x-ray tube may be disposed in a housing of the HV tank that includes oil for HV insulation.

Thus, by disposing the HV tank in the head unit 108 and balancing the weight of the x-ray source system 102 between the head unit 108 and base unit 112, the overall system may be stabilized and also the weight of the overall system may be substantially reduced. Also, the x-ray source system may be easily moved to a desired location/position for scanning the object 106.

Referring to FIG. 2, a block diagram of an x-ray source system of FIG. 1, in accordance with aspects of the present disclosure, is depicted. For ease of understanding, the x-ray source system 200 is described with reference to the components of FIG. 1. The x-ray source system 200 includes a head unit 108 and a base unit 112. It is to be noted that the x-ray source system 200 may include other components, and is not limited to the components shown in FIG. 2.

Further, the head unit 108 may be configured to emit x-rays 104 towards a patient or an object 106 that is under scan. The head unit 108 includes a HV tank 202, an x-ray tube 204, and a collimator 206. In one embodiment, the HV tank 202 may have a housing that includes an x-ray tube 204 along with oil for HV insulation, as depicted in FIG. 2. Also, the HV tank 202 includes a compact voltage multiplier 220 that is configured to receive a low voltage signal from the base unit and generate a high voltage signal based on the received low voltage signal. In one embodiment, the high voltage signal is in a range from about 10 kV to about 200 kV. Further, the high voltage signal is provided to the x-ray tube 202 to emit x-rays 104 toward the object 106. In one example, the high voltage signal may be in a range from about 10 kV to about 200 kV. Further, the emitted x-rays 104 are collimated by the collimator 206 that is disposed at a predefined distance from the x-ray tube 204. Particularly, the collimator 206 may include one or more attenuating units (not shown) that aid in collimating the emitted x-rays 104 and to provide a desired field of view at the object 106 under scan.

In a presently contemplated configuration, the base unit 112 includes low voltage power electronics for providing a low voltage signal to the HV tank 202. Particularly, the base unit 112 includes a power source 208, a rectifier 210, an energy storage unit 212, and an inverter 214. The power source 208 is used for supplying an input voltage to the rectifier 210. In one example, the input voltage may be in a range from about 110 V to about 240 V. In one embodiment, the power source 208 may be disposed external to the x-ray source system 200, and a power cable may be used to couple this external power source to the power electronics, such as the rectifier 210, in the x-ray source system 200.

Further, the rectifier 210 is configured to rectify the input voltage received from the power source 208 into a direct current (DC) voltage. In one example, the DC voltage may be in a range from about 300V to about 500V. This converted DC voltage may be stored in the energy storage unit 212. The exemplary system 300 may have the flexibility of storing the DC voltage within the energy storage unit 212 and using this stored DC voltage when required by the x-ray tube 204 for scanning the object 106. In one embodiment, the converted DC voltage may be provided directly from the rectifier 210 to the inverter 214. Further, the inverter 214 is configured to convert the DC voltage into a high frequency alternating current (AC) voltage. For example, the DC voltage may be converted to an AC voltage that is in a range from about 50 V to 4 kV and having a frequency of about 200 kHz.

In the exemplary system, the inverter 214 in the base unit 112 is coupled to the HV tank 202 through a light weight cable 216. Since the HV tank 202 is disposed within the head unit 108, a low or medium voltage signal is supplied from the base unit 112 to the head unit 108. For supplying such a low or medium voltage signal, a light weight cable 216 of very low thickness may be used between the base unit 112 and the head unit 108. In one example, the light weight cable 216 may have a diameter in a range from about 0.125 inches to about 0.25 inches and a weight of about 0.1 lb to about 0.5 lb. Also, the light weight cable 216 is flexible and easy to move to a desired position, which further aids in moving the head unit 108 to a desired location/position for scanning the object 106.

Thus, by disposing the HV tank 202 in the head unit 108 and using the light weight cable 216 between the head unit 108 and the base unit 112, the weight of the x-ray source system 200 may be substantially reduced. Also, the x-ray source system 200 may be moved to any location/position without much effort from the operator.

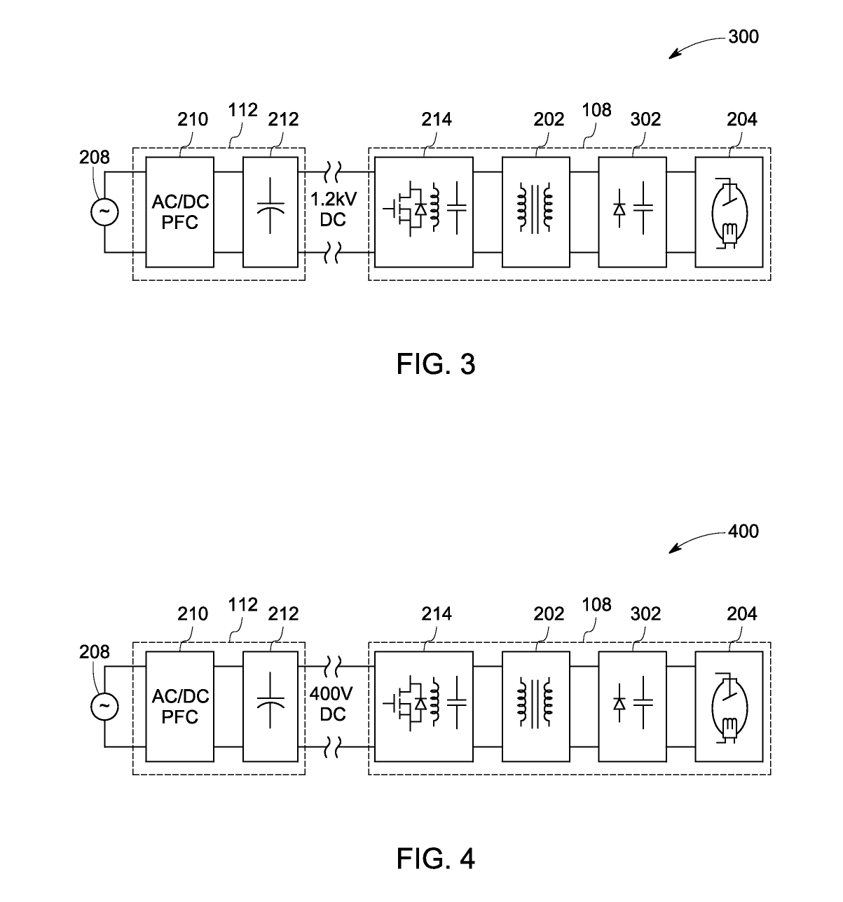

Referring to FIG. 3, a diagrammatical representation of the x-ray source system, in accordance with one embodiment of the present disclosure, is depicted. The x-ray source system 300 is similar to the x-ray source system 200 of FIG. 2 except that the inverter 214 is disposed within the head unit 108. Also, in FIG. 3, a diode unit 302 is used between the HV tank 202 and the x-ray tube 204 to regulate the voltage supplied to the x-ray tube 204. Particularly, the diode unit 302 includes a high voltage diode that receives the AC voltage signal from the HV tank and regulates the received AC voltage signal to a high DC voltage signal. In one example, 6.5 kv silicon carbide (SiC) diode may be used to provide a high DC voltage signal to the x-ray tube 204.

Furthermore, in the embodiment of FIG. 3, the power source 208 is disposed external to the x-ray source system 300, and may be coupled to the rectifier 210 by a power cable. The rectifier may convert the input voltage received from the power source into a DC voltage. Also, the x-ray source system 300 may have a flexibility of storing this DC voltage in the energy storage unit 212 and may be used by the head unit as required.

In the exemplary system, a light weight cable is used to couple the base unit 112 and the head unit 108. This light weight cable is used to convey a DC voltage to the inverter 214 in the head unit 108. In one example, the light weight cable may be used to supply a DC voltage that is in a range from about 50 V to 4 kV from the base unit 112 to the head unit 108.

Moreover, the inverter 214 in the head unit 108 is configured to convert this DC voltage to a high frequency AC voltage. In one example, the inverter may be a 3.3 kv SiC MOS inverter that operates at a frequency range from about 250 kHz to about 800 kHz. Further, this AC voltage is provided to the HV tank 202 to convert into a high voltage signal. In one example, 3 kV-10 kV transformer is used for providing a high voltage signal to the x-ray tube 204 via the diode unit 302.

Referring to FIG. 4, a diagrammatical representation of the x-ray source system, in accordance with another embodiment of the present disclosure, is depicted. The x-ray source system 400 is similar to the x-ray source system 300 of FIG. 3 except that the x-ray source system 300 of FIG. 3 uses a light weight cable to supply a high DC voltage from the base unit 210 to the head unit 108, whereas the exemplary system 400 of FIG. 4 employs a light weight cable to supply a medium DC voltage from the base unit 210 to the head unit 108. Since the medium DC voltage is supplied between the base unit 112 and the head unit 108, the diameter of the light weight cable may be substantially reduced. As the diameter of the cable is reduced, the weight of the light weight cable may also be reduced, which in turn improves the flexibility of the light weight cable.

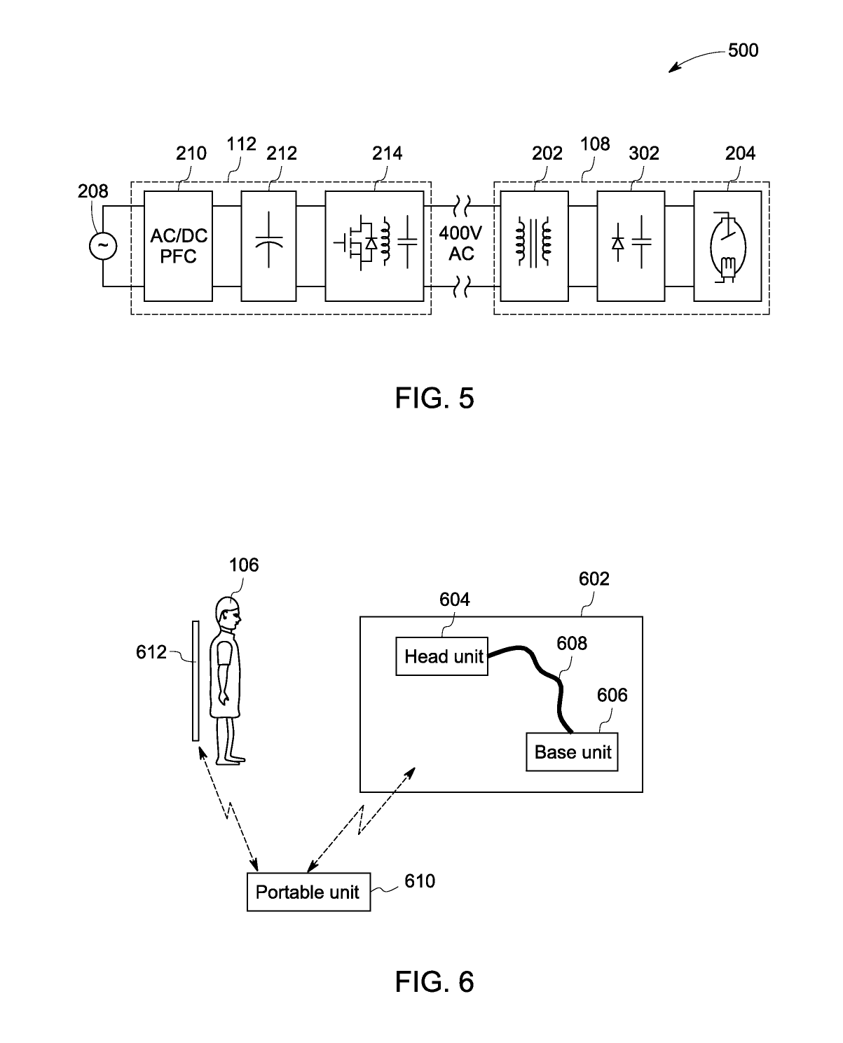

Referring to FIG. 5, a diagrammatical representation of the x-ray source system, in accordance with yet another embodiment of the present disclosure, is depicted. The x-ray source system 500 is similar to the x-ray source system 200 of FIG. 2. In FIG. 5, the inverter operates at a frequency range from about 250 kHz to about 800 khz. For example, a Si-MOS inverter may be used to operate at a frequency range of about 250 kHz and a SiC-MOS inverter may be used to operate at a frequency range of about 400 kHz. Similarly, GaN-MOS inverter may be used to operate at a frequency range of about 800 kHz.

FIG. 6 depicts a diagrammatical representation of an x-ray source system coupled to a portable unit, in accordance with aspects of the present disclosure. The x-ray source system 602 is similar to the x-ray source system 102 of FIG. 1. The X-ray source system 602 includes a head unit 604, a base unit 606, and a low voltage low voltage cable 608. The head unit 604 and the base unit 606 are similar to the head unit 108 and the base unit 112 of FIG. 1. Further, the low voltage cable 608 is a light weight flexible cable that is coupled between the base unit 606 and the head unit 604. Also, the low voltage cable 608 is used to provide a low voltage signal from the base unit 606 to the head unit 604. The low voltage signal may include DC or AC voltage that is in range from about 50 V to 4 kV. It may be noted that the terms “low voltage signal” and “light weight cable” may be used interchangeably in the below description.

In an exemplary embodiment, the x-ray source system 602 may be communicatively coupled to a portable unit 610. The Portable unit 610 may communicate with a detector 612 and the base unit 606 to synchronize the detector 612 with the x-ray source system 602. Particularly, the portable unit 610 may receive an x-ray image from the detector 612 from the detector, and in response, the portable unit 610 may send one or more control signals to the base unit 606. Upon receiving the control signals, the base unit 606 may adjust the light weight x-ray head so that the detector 612 is synchronized with the light weight x-ray head. Also, the portable unit 610 may send the received x-ray image to a remote hospital picture archiving and communication system (PACS) for further processing and/or analyzing of the received x-ray image.

In addition, the portable unit 610 may be used as a remote controller to the base unit 606 or the head unit 604. More specifically, the portable unit 610 may determine at least one parameter of the object 106 based on the received image. The parameter may include at least object/patient size, distance, and alignment of the object. Further, the portable unit 610 may send one or more control signals to the x-ray source system 602 for positioning the head unit 604 to a predetermined position. Particularly, the control signals may be sent to the base unit 606 and/or the head unit 604 to align the head unit 604 to the predetermined position for imaging the object. In one embodiment, the portable unit 610 may be disposed within the base unit 606 and may send the control signals to the head unit 604 via the low voltage cable 608.

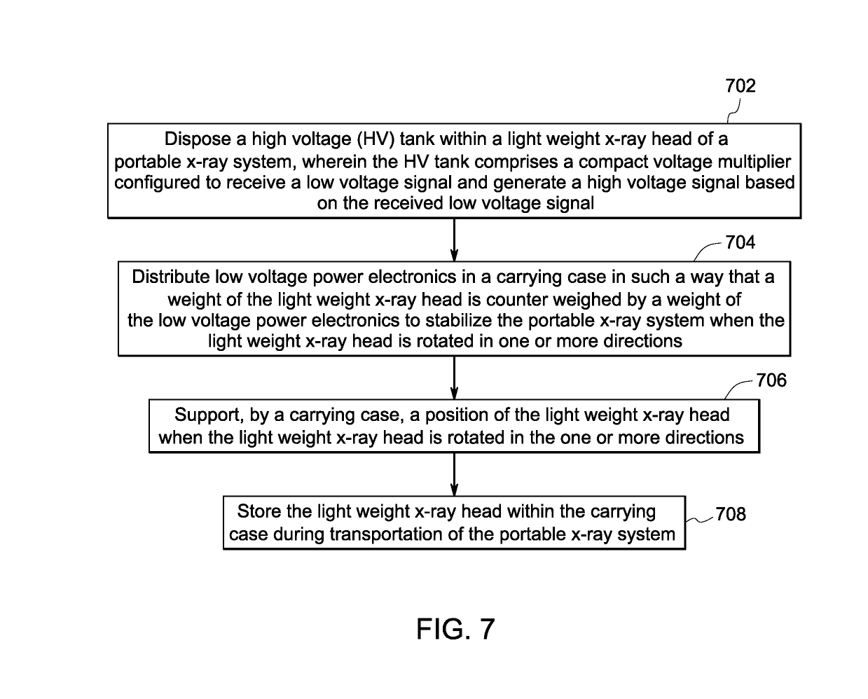

FIG. 7 is a flow chart illustrating a method for balancing a weight of a x-ray source system, in accordance with aspects of the present disclosure. For ease of understanding of the present disclosure, the method is described with reference to the components of FIGS. 1-6. The method begins at step 702, where a high voltage tank 202 is disposed within a head unit 108 of the x-ray source system 102. In one embodiment, the HV tank 202 may be disposed within the head unit 108 to reduce a weight of a portable x-ray source system 102.

In the conventional x-rays source system, the HV tank is disposed in a base unit and coupled to the head unit via a heavy HV cable. This heavy HV cable may be rigidly coupled to the head unit and may have a large diameter, which makes difficult for the operator to move the head unit to a desired position/location for scanning the object. Thus, to overcome these problems/shortcomings, in the exemplary system, the HV tank is disposed within the head unit so that the HV cable and HV connectors may be eliminated from the system 102.

In addition, the HV tank 202 may include a compact voltage multiplier 220 that is configured to receive a low voltage signal and generate a high voltage signal based on the received low voltage signal. In one embodiment, the low voltage signal may be provided from the base unit 112 to the head unit 108 via a low voltage cable or a light weight cable 216, 608.

Additionally, at step 704, low voltage power electronics is distributed in the carrying case or the base unit 112 to balance the weight of the x-ray source system 102. In one example, the low voltage power electronics may include components, such as a rectifier 210, an energy storage unit 212, and an inverter 214. Also, the low voltage power electronics 210, 212, 214 are distributed smartly within a predefined space in the base unit 112. In one example, the predefined space may be referred to as an available space within an x-ray source support frame 110 of the base unit 112. This in turn makes the whole base unit 112 more compact and reduces the size of the portable x-ray system 102. In another example, the predefined space may be any available space between layers of the carrying case 112, a handle of the carrying case 112, inside a rail or any supporting structure of the carrying case 112. Particularly, when the head unit 108 is rotated in one or more directions, the weight of the head unit 108 may imbalance the overall weight of the x-ray source system 102, which in turn destabilizes the x-ray source system 102. Thus, in the exemplary embodiment, the low voltage power electronics 210-214 are distributed in the base unit 112 in such a way that a weight of the head unit 108 is counter weighed by a weight of the low voltage power electronics 210-214 and the x-ray system 102 is stabilized irrespective of the position/direction of the head unit 108.

Furthermore, at step 706, the position of the head unit 108 is supported by the base unit or the carrying case 112 when the head unit 108 is rotated in the one or more directions. Particularly, the base unit 112 includes an x-ray source support frame or positioner 110 that is coupled to the head unit 108. Further, the x-ray source support frame 110 may be extended from the base unit 112 to support the position of the head unit 108.

In addition, at step 708, the head unit 108 may be stored in the base unit 112 during transportation of the x-ray source system. Particularly, the head unit 108 and the x-ray source support frame 110 may be moved into the base unit 112 in such a way that the whole base unit 112 may act as a carrying case for shifting the x-ray source system 102 from one location to another location. Further, during imaging of the object, the stored head unit 108 and the x-ray source support frame 110 may be retrieved from the base unit 112.

Thus, by disposing the HV tank 202 in the head unit 108 and balancing the weight of the x-ray source system 102 between the head unit 108 and base unit 112, the overall system weight may be substantially reduced. Also, the x-ray source system may be easily moved to a desired location/position for scanning the object 106.

The various embodiments of the system and method described hereinabove aid in balancing the weight of the x-ray source system. Also, the weight of the x-ray source system may be substantially reduced. In addition, since a light weight cable is used between the head unit and the base unit, the x-ray source system may be easily moved to any desired location/position for scanning the object.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A portable x-ray system, comprising:
a light weight x-ray head comprising an x-ray tube and a high voltage (HV) tank, wherein the HV tank comprises a compact voltage multiplier configured to receive a low voltage signal and generate a high voltage signal based on the received low voltage signal;
a carrying case comprising low voltage power electronics coupled to the light weight x-ray head through a low voltage cable, and configured to send the low voltage signal to the light weight x-ray head,
wherein the low voltage power electronics is distributed in a predefined space in the carrying case in such a way that a weight of the light weight x-ray head is counter weighed by a weight of the low voltage power electronics to stabilize the portable x-ray system when the light weight x-ray head is rotated in one or more directions, and
wherein the carrying case is configured to support a position of the light weight x-ray head when the light weight x-ray head is rotated in the one or more directions, and store the light weight x-ray head during transportation of the portable x-ray system.

2. The portable x-ray system of claim 1, wherein the carrying case comprises an x-ray source support frame extended from the carrying case and configured to support the position of the light weight x-ray head when the light weight x-ray head is moved in the one or more directions.

3. The portable x-ray system of claim 1, wherein the carrying case comprises at least one of a battery, a wall plug power regulator, an inverter and a power source to provide power to the low voltage power electronics.

4. The portable x-ray system of claim 1, wherein the low voltage cable is configured to supply the low voltage signal comprising at least one of a DC voltage and an AC voltage to the HV tank.

5. The portable x-ray system of claim 4, wherein the at least one of the DC voltage and the AC voltage is in a range from about 50 V to 4 kV.

6. The portable x-ray system of claim 1, further comprising a portable unit communicatively coupled to at least one of the light weight x-ray head and the low voltage power electronics, and configured to send one or more control signal corresponding to imaging of an object.

7. The portable x-ray system of claim 6, wherein the portable unit is comprised in the carrying case.

8. The portable x-ray system of claim 6, wherein the portable unit is remotely coupled to the carrying case.

9. The portable x-ray system of claim 6, wherein the portable unit is communicatively coupled to a detector and configured to receive at least one image from the detector.

10. The portable x-ray system of claim 9, wherein the portable unit is configured to:
determine at least one parameter of the object; and
send the one or more control signals to the light weight x-ray head to align the light weight x-ray head to a predetermined position for imaging the object.

11. The portable x-ray system of claim 9, wherein the portable unit is configured to synchronize the detector with the light weight x-ray head.

12. The portable x-ray system of claim 6, wherein the portable unit is communicatively coupled to a picture archiving and communication system (PACS) and configured to send an x-ray image of the object to the PACS.

13. A method comprising:
disposing a high voltage (HV) tank within a light weight x-ray head of a portable x-ray system, wherein the HV tank comprises a compact voltage multiplier configured to receive a low voltage signal and generate a high voltage signal based on the received low voltage signal;
distributing low voltage power electronics in a carrying case in such a way that a weight of the light weight x-ray head is counter weighed by a weight of the low voltage power electronics to stabilize the portable x-ray system when the light weight x-ray head is rotated in one or more directions;
supporting, by a carrying case, a position of the light weight x-ray head when the light weight x-ray head is rotated in the one or more directions; and
storing the light weight x-ray head within the carrying case during transportation of the portable x-ray system.

14. The method of claim 13, wherein supporting the position of the light weight x-ray head comprises extending an x-ray source support frame from the carrying case to support the position of the light weight x-ray head when the light weight x-ray head is rotated in the one or more directions.

15. The method of claim 13, wherein disposing the high voltage (HV) tank comprises coupling a low voltage cable between the light weight x-ray head and the carrying case to supply the low voltage signal to the HV tank disposed in the light weight x-ray head.

16. The method of claim 15, wherein the low voltage signal comprises at least one of a DC voltage and an AC voltage, wherein the least one of the DC voltage and the AC voltage is in a range from about 50 V to 4 kV.

17. The method of claim 13 further comprising sending one or more control signals from a portable unit to the light weight x-ray head to control imaging of an object.

18. The method of claim 17, wherein sending one or more control signals comprises:
determining at least one parameter of the object;
generating the one or more control signals based on the determined at least one parameter of the object; and
sending the one or more generated control signals to the light weight x-ray head to align the light weight x-ray head to a predetermined position for imaging the object.

19. The method of claim 17 further comprising sending an x-ray image of the object from the portable unit to PACS.

20. The method of claim 13, wherein the high voltage signal is in a range from about 10 kV to 200 kV.
